(19) United States
(12) Patent Application Publication
(10) Pub. No.: US 2020/0163713 A1
(43) Pub. Date: May 28, 2020

(54) CATHETER SYSTEMS AND METHODS FOR ABLATING VARICOSE VEINS

(71) Applicant: CROSSFIRE MEDICAL LLC, Maple Grove, MN (US)

(72) Inventors: Brady HATCHER, Maple Grove, MN (US); Randy BEYREIS, Maple Grove, MN (US)

(73) Assignee: Crossfire Medical LLC, Maple Grove, MN (US)

(21) Appl. No.: 16/611,459

(22) PCT Filed: May 8, 2018
(86) PCT No.: PCT/US2018/031660

§ 371 Date: Nov. 6, 2019

Related U.S. Application Data
(60) Provisional application No. 62/503,160, filed on May 8, 2017.

(51) Int. Cl. A61B 18/14; A61M 25/00; A61M 25/01; A61B 90/00; A61B 18/00

(52) U.S. Cl. CPC A61B 18/1492; A61M 25/0043; A61M 25/0152; A61B 90/06; A61B 2018/00196; A61B 2090/061; A61B 2090/378; A61B 2018/00345; A61M 2025/0008; A61B 2090/3925; A61B 2018/00577; A61B 2090/062; A61M 2025/0047

(57) ABSTRACT

In some embodiments, the disclosure describes a catheter system for applying energy to a vessel, such as a vein. The system may include an elongate catheter shaft having a distal end and a proximal end opposite the distal end, and an energy application device coupled to the distal end of the elongate catheter shaft. The energy application device may be configured to apply energy to the vessel. The system may also include a gauge positioned relative to a working surface. The gauge may be configured to determine a distance that the elongate catheter shaft travels with respect to a treatment zone.

### CROSS-REFERENCE TO RELATED APPLICATIONS

This application claims the benefit and priority of U.S. Provisional Patent Application No. 62/503,160; filed May 8, 2017; and entitled CATHETER SYSTEMS AND METHODS FOR ABLATING VARICOSE VEINS; the entire contents of which are incorporated herein by reference.

### BACKGROUND

### Field of Invention

The present invention relates to catheter systems and methods and more specifically to catheter systems and method for ablating varicose veins.

### Description of Related Art

Varicose veins are veins that suffer from swelling and tortuous lengthening, most often in the legs of a person. Varicose veins may be unattractive and may lead to health complications. Accordingly, many people opt to have varicose veins removed through a common medical procedure, known as an ablation. The ablation procedure involves the insertion of a catheter into a vein in the patient's leg. The catheter is then moved into place whereby it delivers heat energy to the portion of the vein that has become varicose. The heat causes the varicose vein to enclose such that it is no longer externally visible.

Prior art ablation procedures may be tedious and ineffective, because they involve catheter systems that are difficult to navigate through a patient's vein. Even more troublesome, the prior art systems are inaccurate in determining the position of the catheter within the vein. Furthermore, prior art catheter systems often employ activation buttons that are ergonomically incorrect and thereby challenging for the physician to engage. Moreover, the prior art catheter systems may be difficult to manufacture. Thus, there is a need for catheter ablation systems and methods that are easier to navigate, more accurate to determine the position within the vein, employ ergonomically correct components, and are easier to manufacture.

### SUMMARY

The present disclosure describes a catheter system for applying energy to a vessel. The catheter system may include an elongate catheter shaft having a distal end and a proximal end opposite the distal end; an energy application device coupled to the distal end of the elongate catheter shaft, wherein the energy application device is configured to apply energy to the vessel; and a gauge positioned relative to a working surface, wherein the gauge is configured to determine a distance that the elongate catheter shaft travels with respect to a treatment zone.

In some embodiments, the gauge is coupled to the working surface. Also, in some embodiments, the working surface comprises at least one of a patient drape surface, a patient leg surface, a table surface, the elongate catheter shaft, and an introducer sheath that slideably receives the elongate catheter shaft. The gauge may define at least one of a tube, rod, and block. Furthermore, the gauge may define at least one of a card, sticker, label, and template. The gauge may be configured to be coupled to the working surface via a hook and loop fastener. Even still, the gauge may be configured to be coupled to the working surface via adhesive.

Even still, in some embodiments, the card comprises an aperture configured to receive at least one of the introducer sheath and the elongate catheter shaft. The card may comprise a slit located adjacent the aperture. The slit may be configured such that the aperture removably receives at least one of the elongate catheter shaft and the introducer sheath. The gauge may comprise at least one marking interval disposed at a predetermined distance. The gauge may even comprise at least one color disposed on the at least one marking interval. The energy application device may define a first length and the at least one marking interval is less than or equal to the first length. The treatment zone may define a second length and the at least one marking interval may be less than or equal to the second length.

The gauge may be slideably coupled to the working surface. The gauge may comprise a sliding object that slideably receives at least one of the elongate catheter shaft and the introducer sheath. In some embodiments, the energy application device may define a first length and the sliding object defines a predetermined length that is less than or equal to the first length. Even still, in some embodiments, the treatment zone may define a second length and the sliding object defines a predetermined length that is less than or equal to the second length. The sliding object may define an overall length that is approximately equal to 7 centimeters.

In some embodiments, the sliding object comprises a hollow inner portion that slideably receives the elongate catheter shaft. Accordingly, the sliding object may slide along a first direction that is parallel to the elongate catheter shaft. In some embodiments, the sliding object frictionally slides with respect to the elongate catheter shaft along the first direction. The friction may be enough such that the sliding object does not move with respect to the elongate catheter shaft under a force greater than or equal to gravity. In some embodiments, the sliding object slides along a second direction that is perpendicular to the elongate catheter shaft. The sliding object may clippably couple to the elongate catheter shaft along the second direction.

The sliding object may comprise a variable friction mechanism, whereby when the variable friction mechanism is depressed the sliding object slides with respect to the elongate catheter shaft, and when the variable friction mechanism is not depressed the sliding object does not slide with respect to the elongate catheter shaft. The variable friction mechanism may comprise a spring loaded clamping device.

The disclosure also includes a catheter system for applying energy to a vessel. The catheter system may include an elongate catheter shaft having a distal end and a proximal end opposite the distal end; an energy application device coupled to the distal end of the elongate catheter shaft, wherein the energy application device is configured to apply energy to the vessel; and one or more echogenic features disposed at predetermined lengths along the elongate catheter shaft. The one or more of echogenic features may be arranged and configured to allow a physician to view a position within the vessel under ultrasound.

The energy application device may define a first length and each of the echogenic features may be spaced apart by a spacing length. The spacing length may be a predetermined multiple of the first length. The multiple may be less than or equal to 1. The spacing length may be equal to the first length less a decrement that is between 1% and 15% of the first length. The one or more echogenic features may define one or more echogenic bands that each surround a portion of the elongate catheter shaft. The one or more echogenic features may comprise a coiled material. As well, the one or more echogenic features may comprise a roughened surface. Even still, the one or more echogenic features may comprise a solid material. Furthermore, the one or more echogenic features comprises a porous material.

In some embodiments, the one or more echogenic features comprises at least one fully radial echogenic feature that surrounds the elongate catheter shaft. In some embodiments, the one or more echogenic features comprises at least one partially radial echogenic feature that partially surrounds the elongate catheter shaft. Even still, in some embodiments, the one or more echogenic features comprises at least one fully radial echogenic feature that surrounds the elongate catheter shaft and at least one partially radial echogenic feature that partially surrounds the elongate catheter shaft. In some embodiments, the elongate catheter shaft defines a presence of echogenicity and the one or more echogenic features defines an absence of echogenicity.

The disclosure also includes methods for determining a position of a catheter system with respect to a second treatment zone of a vessel, wherein the second treatment zone occurs after a first treatment zone. Methods may comprise inserting an elongate catheter shaft into a vessel of a patient, wherein the elongate catheter shaft has a distal end and a proximal end opposite the distal end, and wherein an energy application device is coupled to the distal end of the elongate catheter shaft, the energy application device comprising echogenic features. Methods may also include determining a position of the energy application device with respect to a second treatment zone of the vessel via one or more echogenic features viewed under ultrasound, wherein the one or more echogenic features are each disposed at predetermined lengths along the elongate catheter shaft. Methods may even include positioning the energy application device in the second treatment zone of the vessel and applying energy to the second treatment zone of the vessel via the energy application device.

Methods may include moving the energy application device away from the second treatment zone of the vessel. Even still, methods may include determining a third position of the energy application device with respect to a third treatment zone of the vessel via the one or more echogenic features viewed under ultrasound. Methods may also include positioning the energy application device in the third treatment zone of the vessel and applying energy to the third treatment zone of the vessel via the energy application device.

The energy application device may define a first length and each of the one or more echogenic features may be spaced apart by a spacing length. The spacing length may be less than or equal to the first length. In some embodiments, the first position of the energy application device is located less than or equal to the spacing length with respect to the second treatment zone of the vessel. The spacing length may be a predetermined multiple of the first length. The multiple may be less than or equal to 1. Even still, the spacing length is equal to the first length less a decrement that is between 1% and 15% of the first length.

The disclosure also includes a catheter system for applying energy to a vessel. The catheter system may include an energy application device configured to apply energy to the vessel and an elongate catheter shaft having a distal end and a proximal end opposite the distal end, wherein the energy application device is coupled to the distal end of the elongate catheter shaft. The elongate catheter shaft may include an inner tube disposed within an internal portion of the elongate catheter shaft and an outer tube that substantially surrounds the inner tube. The inner tube may comprise etched polytetrafluoroethylene (PTFE).

In some embodiments, the catheter system further comprises a middle tube that substantially surrounds the inner tube. The middle tube may comprise a thermoplastic. In some embodiments, the middle tube comprises Pebax.

The outer tube may comprise a thermoplastic. In some embodiments, the outer tube comprises Pebax.

In some embodiments, the catheter system further comprises a hub coupled to the proximal end of the elongate catheter shaft. The hub may have a feature configured to activate the energy application device. In some embodiments, the outer tube may continuously extend from the distal end of the elongate catheter shaft to the hub at the proximal end of the elongate catheter shaft.

The disclosure also includes a catheter system for applying energy to a vessel. The catheter system may include an elongate catheter shaft having a distal end and a proximal end opposite the distal end and an energy application device coupled to the distal end of the elongate catheter shaft, wherein the energy application device is configured to apply energy to the vessel. The catheter system may include a movable tip coupled to a distal end of the energy application device, wherein the movable tip is arranged and configured to be at least one of curved and angled with respect to the elongate catheter shaft. The catheter system may also include a stylet disposed within an internal portion of the elongate catheter shaft, wherein the stylet is arranged and configured to move between a proximal end point and a distal end point within the elongate catheter shaft.

In some embodiments, the proximal end point and the distal end point are located a predetermined distance from each other such that the stylet is free to move within the elongate catheter shaft between the proximal end point and the distal end point. When the stylet is moved to the proximal end point the stylet may be completely detached from the internal portion of the elongate catheter shaft. When the stylet is moved towards the distal end point the energy application device may become straightened. Accordingly, when the stylet is moved towards the proximal end point the energy application device may be at least one of curved and angled. The stylet may be arranged and configured to be secured in place with respect to the energy application device.

Even still, the disclosure includes a catheter system for applying energy to a vessel. The catheter system may include an elongate catheter shaft having a distal end and a proximal end opposite the distal end, and an energy application device coupled to the distal end of the elongate catheter shaft, wherein the energy application device is configured to apply energy to the vessel. The catheter system may include a stylet disposed within an internal portion of the elongate catheter shaft, wherein the stylet is arranged and configured to allow the elongate catheter shaft to be pushed into and pulled out of the vessel.

The disclosure also includes a catheter system for applying energy to a vessel. The catheter system may comprise an elongate catheter shaft having an internal portion, a distal end, and a proximal end opposite the distal end, and an energy application device coupled to the distal end of the elongate catheter shaft, wherein the energy application device is configured to apply energy to the vessel. The catheter system may include a hub coupled to the proximal end of the elongate catheter shaft, wherein the hub is arranged and configured to lay flat on a working surface. The catheter system may also include a push button communicatively coupled to the energy application device, wherein the push button is configured to activate the energy application device.

In some embodiments, the push button is coupled to the hub. The catheter system may also include a push button extension communicatively coupled to the energy application device, wherein the push button extension is located remotely with respect to the hub and push button. The push button extension may comprise a wireless remote control.

When the hub and push button move, the push button extension may be arranged and configured to remain in a fixed position with respect to the hub and push button. The push button may be located remotely with respect to the hub. When the hub moves, the push button may be arranged and configured to remain in a fixed position with respect to the hub. The hub and push button may be sized and shaped to be ergonomically fitted to a human hand. The energy application device may be electrically coupled to the hub.

In some embodiments, the catheter system may include a proximal connector that is electrically coupled to the energy application device and the hub. The energy application device and the proximal connector may be electrically coupled via a continuous uninterrupted electrical connection. The continuous uninterrupted electrical connection may be a continuous wire.

The disclosure also includes a catheter system for applying energy to a vessel. The catheter system may include an elongate catheter shaft having an internal portion, a distal end, and a proximal end opposite the distal end. The system may also include an energy application device coupled to the distal end of the elongate catheter shaft, wherein the energy application device is configured to apply energy to the vessel. As well, the system may include a hub coupled to the proximal end of the elongate catheter shaft. The hub may have a push button configured to activate the energy application device and a luer port arranged and configured to receive a stylet.

In some embodiments, the catheter system further comprises a stylet disposed within an internal portion of the elongate catheter shaft. The system may further include a one-way valve in fluid communication with the luer port. The one-way valve may be coupled to the stylet whereby the one-way valve may be arranged and configured to allow a fluid to be discharged into the internal portion of the elongate catheter shaft.

Even still, the disclosure includes methods for prepping an inner lumen located within an elongate catheter shaft. Methods may include coupling a syringe to a luer port in fluid communication with the inner lumen, pushing saline, via the syringe, into the inner lumen to remove air from the inner lumen, and maintaining fluid stasis, via a one-way valve within the luer port, between the syringe and the inner lumen. Methods may include inserting a stylet into the luer port and into the elongate catheter shaft.

In some embodiments, the method includes fixing the stylet with respect to the luer port. Methods may include fixing the stylet with respect to the luer port and rotating a portion of the luer port to thereby lock the stylet with respect to the luer port. In some embodiments, the method includes pushing saline through the one-way valve into the inner lumen while the stylet is fixed with respect to the luer port.

The disclosure also includes a catheter system for applying energy to a vessel. The catheter system may include an elongate catheter shaft having an internal portion, a distal end, and a proximal end opposite the distal end. The system may include an energy application device coupled to the distal end of the elongate catheter shaft, wherein the energy application device is configured to apply energy to the vessel. As well, the catheter system may include a hub coupled to the proximal end of the elongate catheter shaft and a power cord coupled to the hub and electrically coupled to the energy application device.

The power cord may comprise a braided cover that at least partially surrounds the power cord. The braided cover may be arranged and configured to provide increased tensile strength. Even still, the power cord may comprise a tethered fiber disposed along an inner portion of the power cord. The tethered fiber may be arranged and configured to provide increased tensile strength.

Other objects and advantages of the embodiments herein will become readily apparent from the following detailed description taken in conjunction with the accompanying drawings. The embodiments described above include many optional features and aspects. Features and aspects of the embodiments can be combined.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments.

FIG. 1 illustrates a catheter system, according to some embodiments.

FIG. 2 illustrates a catheter system and a treatment zone, according to some embodiments.

FIG. 3 illustrates a catheter system having an introducer sheath, according to some embodiments.

FIG. 4 illustrates a gauge having an aperture and a slit, according to some embodiments.

FIGS. 5-8 illustrate gauges and working surfaces, according to some embodiments.

FIG. 9 illustrates a catheter system having a sliding object, according to some embodiments.

FIG. 10 illustrates a side-view of a sliding object, according to some embodiments.

FIG. 11 illustrates an elongate catheter shaft and a sliding object, according to some embodiments.

FIG. 12 illustrates a catheter system having echogenic features, according to some embodiments.

FIG. 13 illustrates a side-view of a fully radial echogenic feature, according to some embodiments.

FIG. 14 illustrates a side-view of a partially radial echogenic feature, according to some embodiments.

FIG. 15 illustrates a flow chart for determining a position of a catheter system, according to some embodiments.

FIG. 16 illustrates a side-view of an elongate catheter shaft, according to some embodiments.

FIG. 17 illustrates a catheter system having a hub and a push button, according to some embodiments.

FIG. 18 illustrates a catheter system having a movable tip, according to some embodiments.

FIG. 19 illustrates a catheter system having a movable tip in a curved state, according to some embodiments.

FIG. 20 illustrates a catheter system having a movable tip in an angled state, according to some embodiments.

FIG. 21 illustrates a catheter system having a movable tip in a straightened state, according to some embodiments.

FIG. 22 illustrates an elongate catheter shaft, according to some embodiments.

FIG. 23 illustrates a catheter system having a movable tip in a curved or angled state, according to some embodiments.

FIG. 24 illustrates a catheter system having a movable tip in a straightened state, according to some embodiments.

FIG. 25 illustrates a catheter system having a movable tip in a curved or angled state, according to some embodiments.

FIG. 26 illustrates a catheter system having a movable tip in a straightened state, according to some embodiments.

FIGS. 27 and 28 illustrate a catheter system having a hub, according to some embodiments.

FIG. 29 illustrates a catheter system having a push button, according to some embodiments.

FIGS. 30-32 illustrate a catheter system having a hub and a power cord, according to some embodiments.

FIGS. 33 and 34 illustrate catheter systems, according to some embodiments.

FIG. 35 illustrates a flow chart for prepping an inner lumen located within an elongate catheter shaft, according to some embodiments.

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Additionally, reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

### LIST OF REFERENCE NUMERALS

10—Catheter system
12—Elongate catheter shaft
14—Distal end
16—Proximal end
18—Energy application device
20—Treatment zone
21—Introducer sheath
22—Working surface
24—Gauge
26—Aperture
28—Slit
30—At least one marking interval
32—First length
34—Sliding object
36—Hollow inner portion
38—Variable friction mechanism
40—Echogenic feature
42—Spacing length
44—Inner tube
46—Middle tube
48—Outer tube
50—Hub
52—Button
54—Movable tip
56—Distal tip
60—Stylet
62—Proximal end point
64—Distal end point
66—Predetermined distance
68—Push button extension
70—Proximal connector
72—Luer port
74—One-way valve
76—Power cord

### INTRODUCTION

Current catheter systems and methods for treating varicose veins have numerous shortcomings. Accordingly, the present disclosure addresses many of these problems and offers solutions to remedy the deficiencies. The solutions provided endeavor to not only enhance patients' treatment experience and improve physicians' ability to navigate and effectively treat varicose veins, but also to increase manufacturers' ability to efficiently produce the catheter systems.

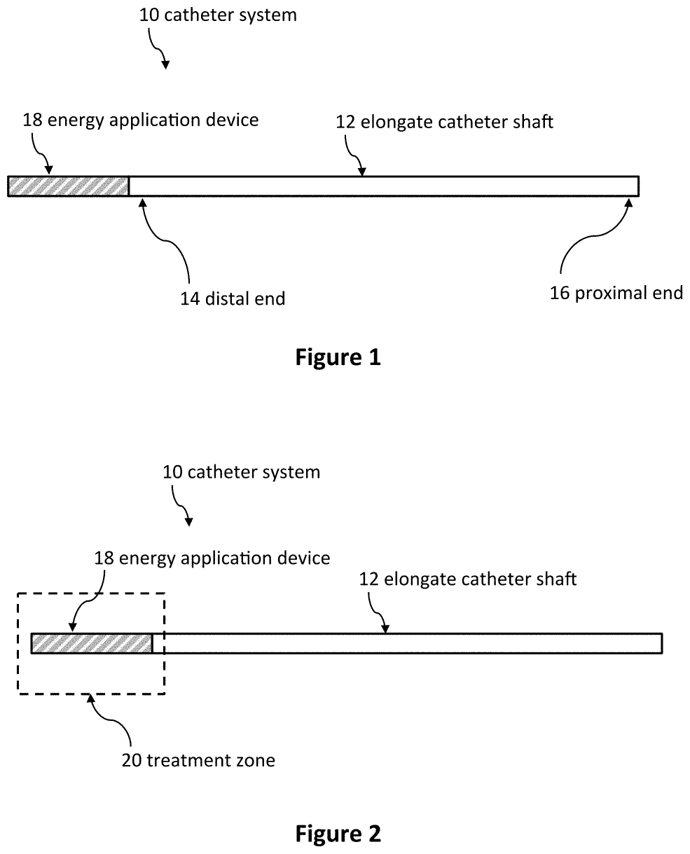

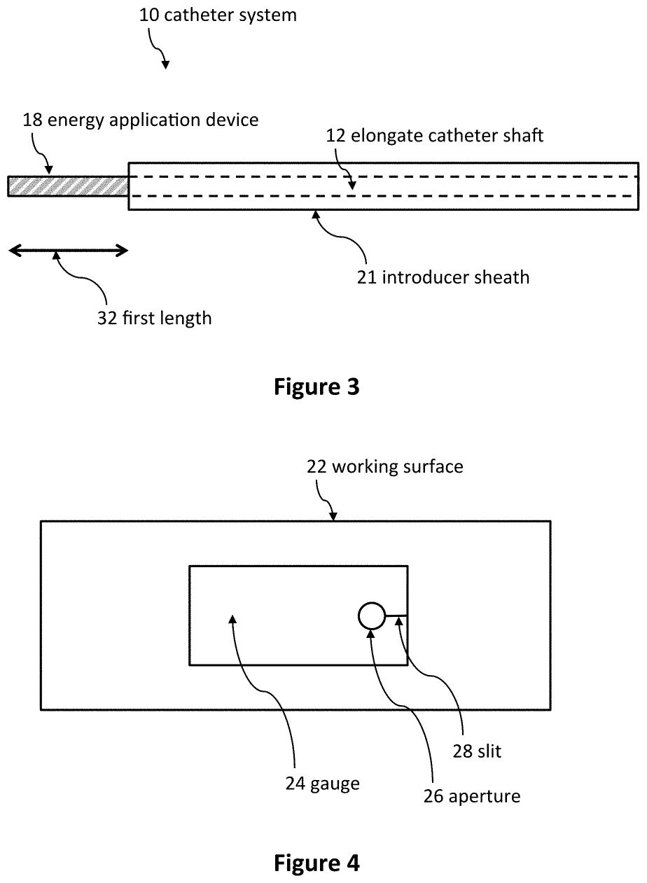

FIGS. 1 and 2 illustrate a catheter system 10 for applying energy to a vessel. The catheter system 10 may include an elongate catheter shaft 12 having a distal end 14 and a proximal end 16 opposite the distal end 14. The system 10 may also include an energy application device 18 coupled to the distal end 14 of the elongate catheter shaft 12. In various embodiments, the energy application device 18 is configured to apply energy to the treatment zone 20 within the vessel. Applying energy to the vessel may cause collagen denaturation and shrinkage such that the vessel diameter is substantially reduced and the vessel wall becomes thickened. Oftentimes, the end result is a vessel filled with fibrin whereby fluid can no longer flow through the vessel. As shown in FIG. 3, and other figures to follow, the catheter system 10 may include additional components, such as an introducer sheath 21 that slideably receives the elongate catheter shaft 12.

### Index Marking Embodiments

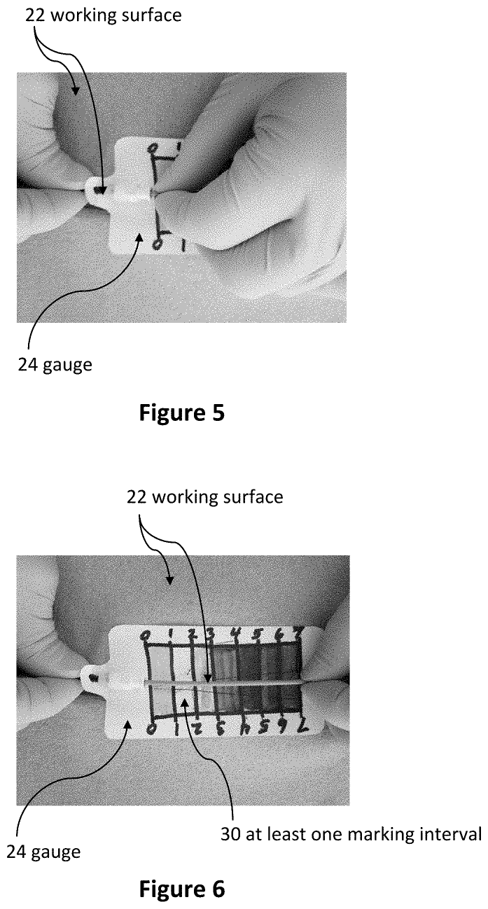

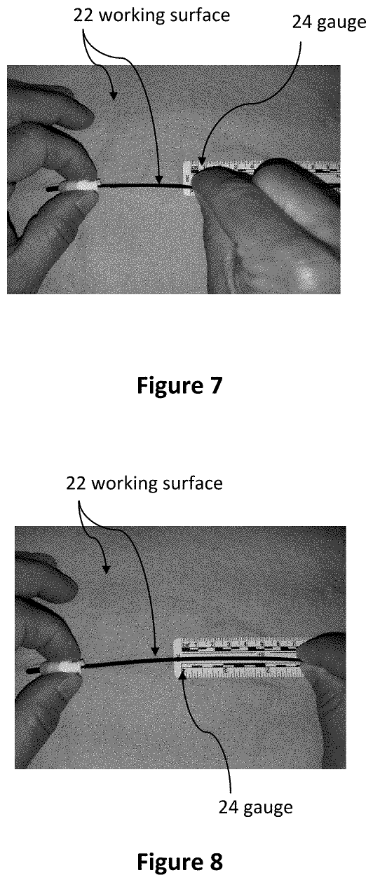

With reference to FIGS. 4-8, the catheter system 10 may include features to assist a physician in determining any such desired measurement for how far to move the catheter system 10 when applying energy to various treatment zones 20. For example, the catheter system 10 may include a gauge 24 positioned relative to a working surface 22. The gauge 24 may be configured to determine a distance that the elongate catheter shaft 12 travels with respect to a treatment zone 20. The gauge 24 may be easier for the physician to quickly and accurately identify a measurement, as opposed to the tiny marks on prior art catheter shafts. Prior art systems oftentimes force the physician to eyeball the movement distance, or measurement, which may result in under treatment or over treatment of the vessel.

The gauge 24 may comprise any type of device arranged and configured to determine distance, such as a tube, rod, and/or block. In some embodiments, the gauge 24 comprises a card, sticker, label, and/or template. The gauge 24 may include an aperture 26 and a slit 28 arranged and configured to removably receive at least one of the elongate catheter shaft 12 and the introducer sheath 21. Accordingly, the gauge 24 may slide along the introducer sheath 21 and/or elongate catheter shaft 12, via the aperture 26. In this regard, the physician may move the gauge 24 along as he or she performs various treatments whereby the gauge 24 may be used to determine how far to move the catheter system 10 for such treatments.

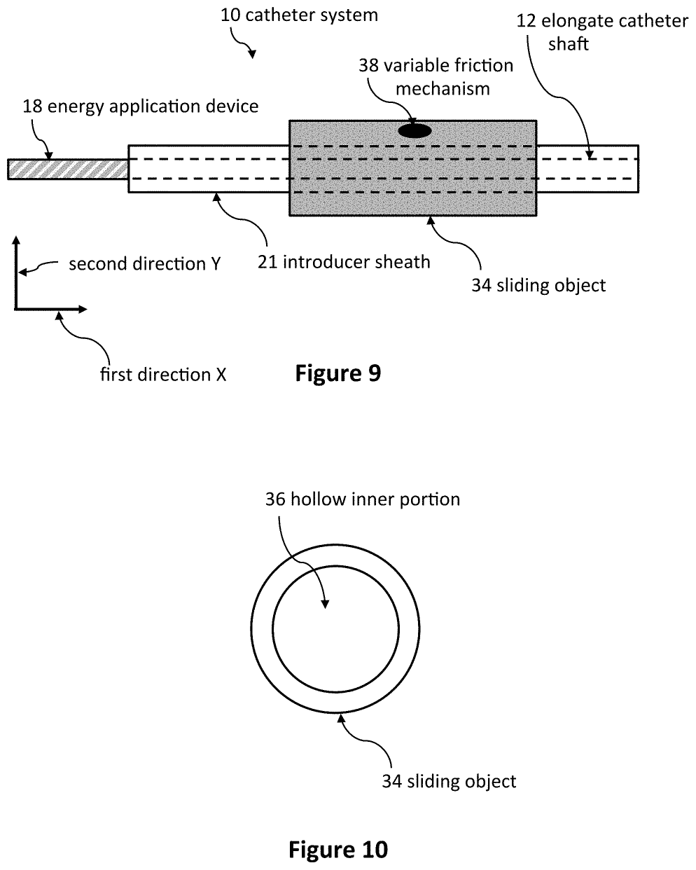

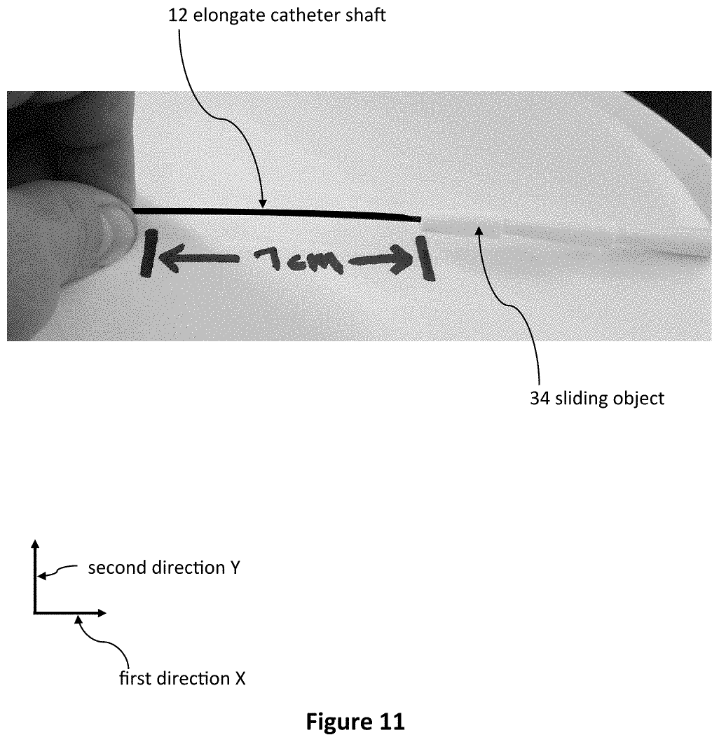

Now, with reference to FIGS. 9-11, the gauge 24 may comprise a sliding object 34 that slideably receives at least one of the elongate catheter shaft 12 and the introducer sheath 21. The sliding object 34 may comprise a hollow inner portion 36 that slideably receives the elongate catheter shaft 12. The sliding object 34 may thereby slide along a first direction X that is parallel to the elongate catheter shaft 12. Alternatively, in some embodiments the sliding object 34 slides along a second direction Y that is perpendicular to the elongate catheter shaft 12.

The sliding object 34 may slide with little to no friction or with friction along the first direction X along the elongate catheter shaft 12. The friction may be enough such that the sliding object 34 does not move with respect to the elongate catheter shaft 12 under a force greater than or equal to gravity. The sliding object 34 may also include features to control the amount of friction. Accordingly, the sliding object 34 may include a variable friction mechanism 38 that is arranged and configured to increase and/or decrease the amount of friction. In some embodiments, when the variable friction mechanism 38 is depressed the sliding object 34 may slide with respect to the elongate catheter shaft 12. When the variable friction mechanism 38 is not depressed, the sliding object 34 does not slide with respect to the elongate catheter shaft 12. In some embodiments, the variable friction mechanism 38 comprises a button and a spring-loaded clamping device that applies friction to the elongate catheter shaft 12.

In some embodiments, the sliding object 34 may engage the elongate catheter shaft 12 by clippably coupling to the elongate catheter shaft 12 along the second direction Y. Alternatively, the sliding object 34 may engage the elongate catheter shaft 12 by slideably receiving the distal end 14 or the proximal end 16 of the elongate catheter shaft 12 along the first direction X. Generally, it should be appreciated that the sliding object 34 may engage the elongate catheter shaft using any such means whereby the sliding object is able to substantially surround the elongate catheter shaft 12.

As shown in FIG. 6, to further assist the physician in determining the distance that the elongate catheter shaft 12 travels with respect to a treatment zone 20, the gauge 24 may include at least one marking interval 30 disposed at a predetermined distance. Accordingly, the physician may use the marking interval 30 to determine how far to move the catheter system 10. With continued reference to FIG. 6, to further enhance the physician's ability to determine the distance, the gauge 24 may include at least one color disposed on the at least one marking interval 30. The at least one color shall provide enough contrast and be dramatic enough that the physician is able to easily distinguish and locate the desired marking interval (e.g. bright colors, or colors on adjacent marking intervals that are quite different).

In some embodiments, the gauge 24 is coupled to the working surface 22, while in some embodiments, the gauge 24 is decoupled from the working surface 22. It should be appreciated that in any such embodiments, the gauge 24 may be permanently or temporarily fixed to the working surface 22. For example, the gauge 24 may be configured to be coupled to the working surface 22 via a hook and loop fastener. Even still, the gauge 24 may be configured to be coupled to the working surface 22 via adhesive. However, in some embodiments, the gauge 24 is able to slide along the working surface 22. Furthermore, it should be appreciated that the working surface 22 may comprise any device or article associated with the catheter system 10 and/or the ablation process, such as a patient drape surface, a patient leg surface, a table surface, the elongate catheter shaft 12, and the introducer sheath 21.

With reference back to FIG. 3, the energy application device 18 may define a first length 32. The at least one marking interval 30 may be less than or equal to the first length. Additionally, the treatment zone 20 may define a second length 33 and, in some embodiments, the at least one marking interval 30 may be less than or equal to the second length 33. In some embodiments, the sliding object 34 defines a predetermined length that is less than or equal to the first length 32. As well, in some embodiments, the sliding object 34 defines a predetermined length that is less than or equal to the second length 33.

In some embodiments, the first length 32, second length 33, and/or sliding object 34 are less than or equal to 7 centimeters. In some embodiments, the first length 32, second length 33, and/or sliding object 34 are greater than or equal to 7 centimeters. Generally, it should be appreciated that the first length 32, second length 33, and/or sliding object 34 may define any length.

### Echogenic Feature Embodiments

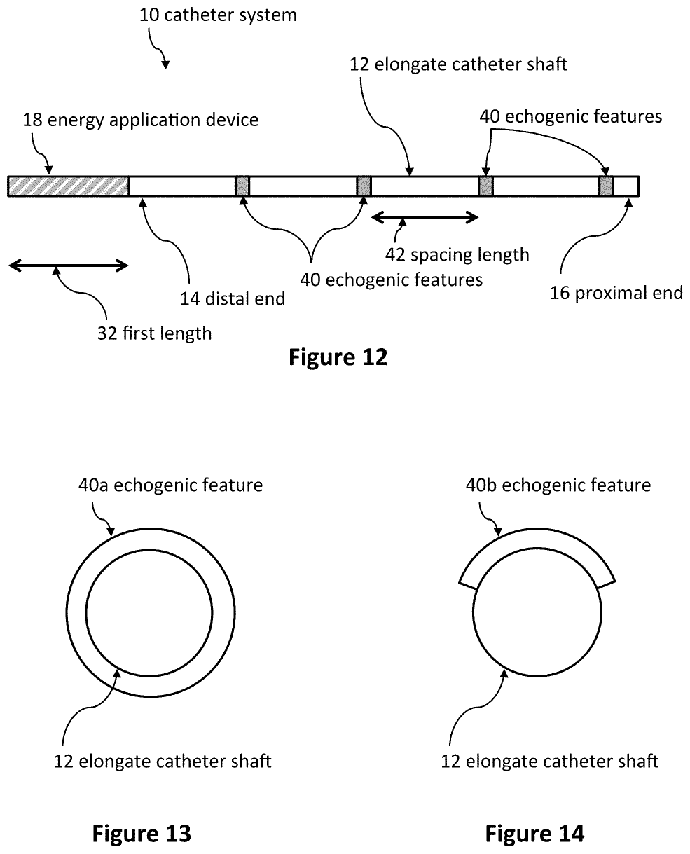

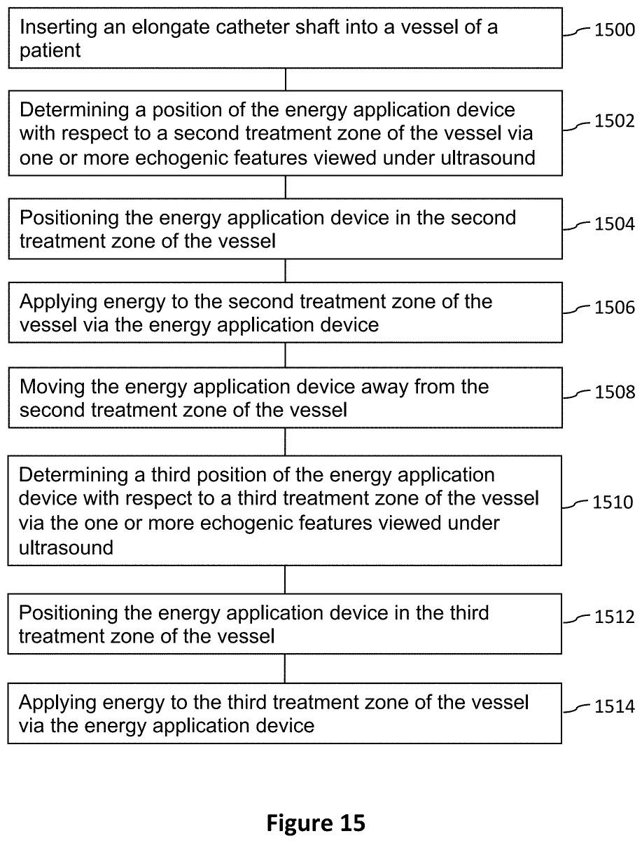

As shown in FIG. 12, the catheter system 10 may include one or more echogenic features 40 disposed at predetermined lengths along the elongate catheter shaft 12. The echogenic features may allow the physician to view a position of the catheter system 10 within the vessel under ultrasound. As previously stated, the energy application device 18 may define a first length 32. Each of the echogenic features 40 may be spaced apart by a spacing length 42. In some embodiments, the spacing length 42 is a predetermined multiple of the first length 32. The multiple may be less than or equal to 1. In some embodiments, the spacing length 42 is equal to the first length 32 less a decrement that is between 1% and 15% of the first length 32. Generally, it should be appreciated that the spacing length 42 may be sized directly or indirectly with respect to the first length 32.

The one or more echogenic features 40 may define one or more echogenic bands that each surround a portion of the elongate catheter shaft 12. As shown in FIG. 13, the one or more echogenic features 40 may include at least one fully radial echogenic feature that surrounds the entire elongate catheter shaft. Alternatively, as shown in FIG. 14, the one or more echogenic features 40 may include at least one partially radial echogenic feature that partially surrounds the elongate catheter shaft. The catheter system 10 may include either all fully radial echogenic features 40a, all partially radial echogenic features 40b, or a mix of one or more fully radial echogenic features 40a and one or more partially radial echogenic features 40b.

In some embodiments, a majority of the elongate catheter shaft 12 defines a presence of echogenicity and the one or more echogenic features 40 defines an absence of echogenicity. In this regard, the one or more echogenic features 40 may be visible under ultrasound because of the presence or absence of echogenicity and the corresponding remainder of the elongate catheter shaft 12 having the opposite. In other words, if the one or more echogenic features 40 has a presence of echogenicity and the corresponding remainder of the elongate catheter shaft 12 has an absence of echogenicity, then the one or more echogenic features 40 may be visible under ultrasound because of the contrast with the remainder of the elongate catheter shaft 12. Alternatively, if the one or more echogenic features 40 has an absence of echogenicity and the corresponding remainder of the elongate catheter shaft 12 has a presence of echogenicity, then again, the one or more echogenic features 40 may be visible under ultrasound because of the contrast with the remainder of the elongate catheter shaft 12.

The one or more echogenic features 40 may define any type of material and/or define any type of surface finish. In some embodiments, the one or more echogenic features 40 includes a coiled material. Additionally, in some embodiments, the one or more echogenic features 40 includes a roughened surface. Even still, the one or more echogenic features 40 may include a solid material, while in some embodiments the one or more echogenic features 40 includes a porous material.

This disclosure also includes methods of using the echogenic features 40 to determine a position of the catheter system 10 with respect to a second treatment zone of a vessel. It should be appreciated that the second treatment zone occurs after a first treatment zone. The methods may include inserting an elongate catheter shaft 12 into a vessel of a patient (at step 1500) and determining a position of the energy application device 18 with respect to a second treatment zone of the vessel via one or more echogenic features 40 viewed under ultrasound (at step 1502). Methods may also include positioning the energy application device 18 in the second treatment zone of the vessel (at step 1504) and applying energy to the second treatment zone of the vessel via the energy application device 18 (at step 1506). Accordingly, methods may include moving the energy application device 18 away from the second treatment zone of the vessel (at step 1508).

Methods may thereby include determining a third position of the energy application device 18 with respect to a third treatment zone of the vessel via the one or more echogenic features 40 viewed under ultrasound (at step 1510). As well, methods may include positioning the energy application device 18 in the third treatment zone of the vessel (at step 1512) and applying energy to the third treatment zone of the vessel via the energy application device 18 (at step 1514).

### Coil and Catheter Tube Embodiments

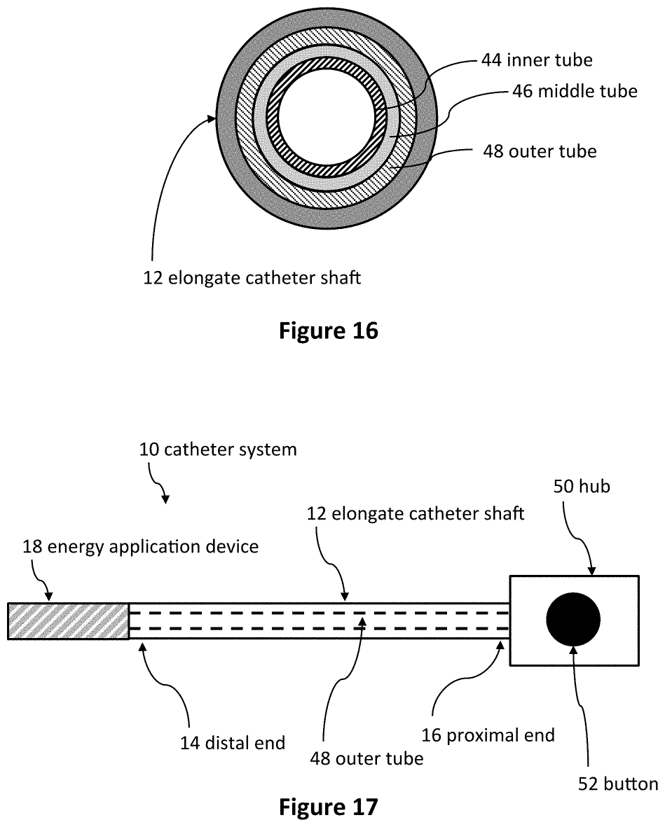

As shown in FIG. 16, the elongate catheter shaft 12 may include one or more tubes or lumens disposed along the internal portion of the elongate catheter shaft 12. In some embodiments, the elongate catheter shaft 12 includes an inner tube 44 disposed within an internal portion of the elongate catheter shaft 12, a middle tube 46 that substantially surrounds the inner tube 44, and an outer tube 48 that substantially surrounds the inner tube 44 and the middle tube 46. Any of the tubes 44, 46, 48 may comprise a thermoplastic. In some embodiments, the inner tube 44 comprises etched polytetrafluoroethylene (PTFE), and the middle tube 46 and the outer tube 48 comprise Pebax.

Such materials may allow the elongate catheter shaft 12 and the tubes 44, 46, 48 to be more lubricious to thereby improve guide wire movement within the internal portion of the elongate catheter shaft 12. As well, the thermoplastic, PTFE, and/or Pebax may result in improved flexibility and improved manufacturability because the materials may be more bondable.

As illustrated in FIG. 17, in some embodiments, the catheter system 10 may include a hub 50 coupled to the proximal end 16 of the elongate catheter shaft 12. The hub 50 may have a feature, such as a button 52, configured to activate the energy application device 18. The outer tube 48 may continuously extend from the distal end 14 of the elongate catheter shaft 12 to the hub 50 at the proximal end 16 of the elongate catheter shaft 12. This may result in improved pushability in a proximal region of the elongate catheter shaft 12 when the physician is pushing the catheter system 10 into a vessel of a patient.

### Movable Tip Embodiments

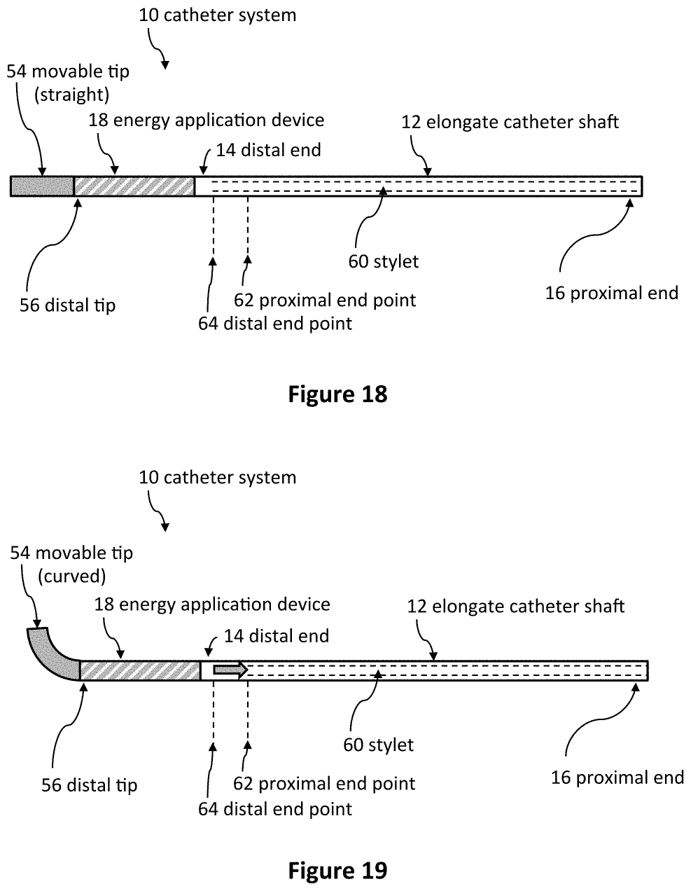

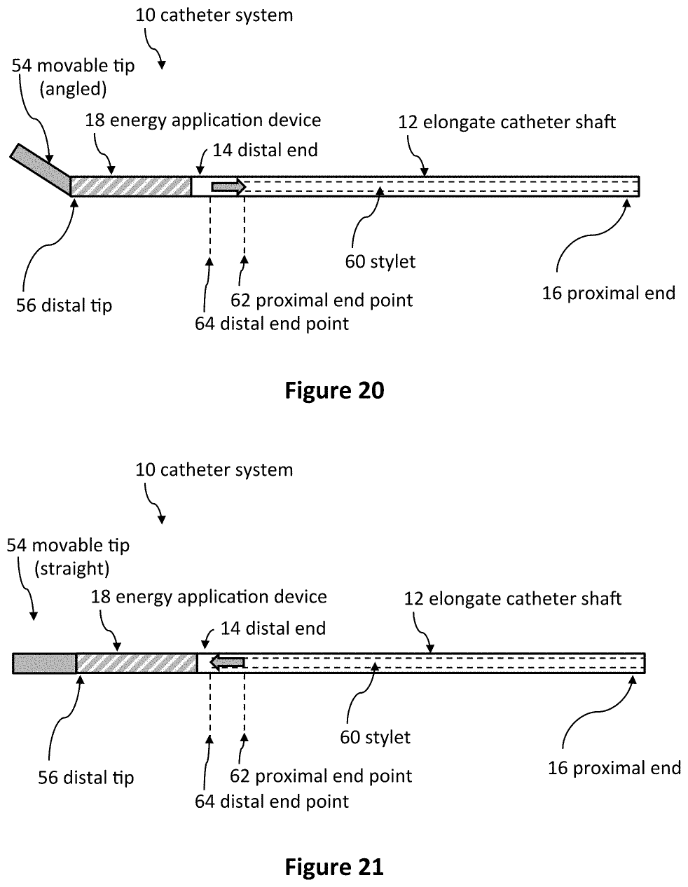

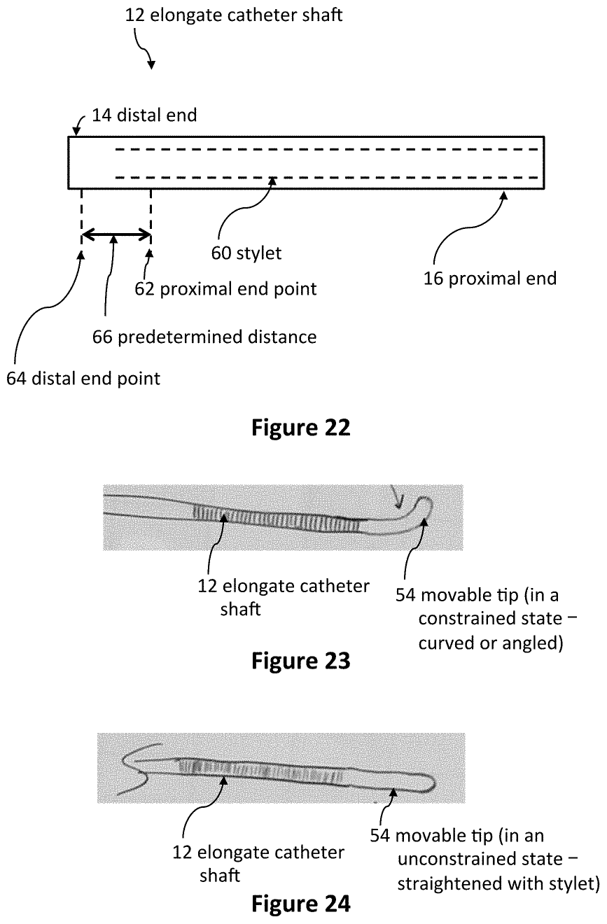

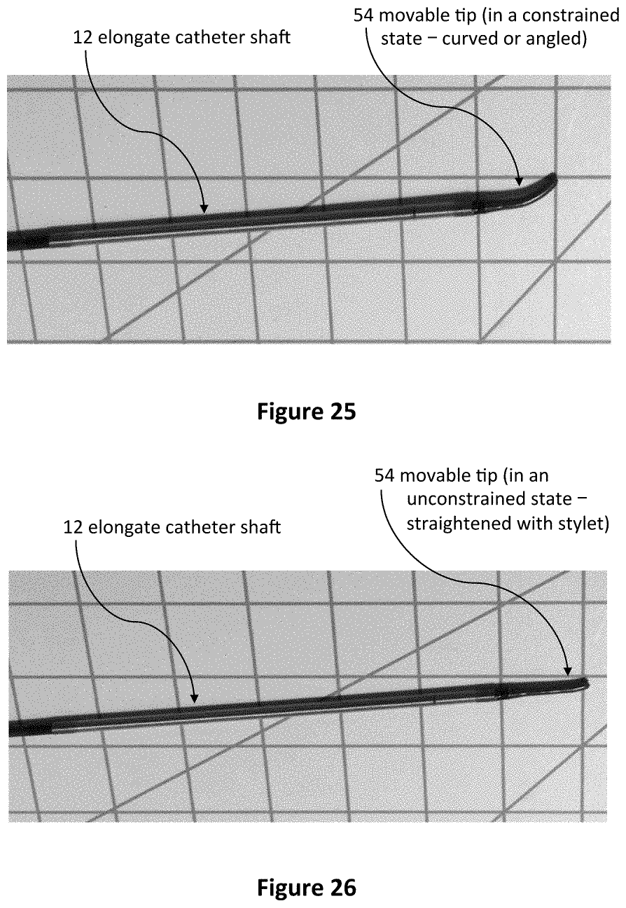

With reference to FIGS. 18-26, the catheter system 10 may include a movable tip 54 coupled to a distal tip 56 of the energy application device 18. The movable tip may be arranged and configured to define a variety of shapes, such as curved, angled, and/or straight, with respect to the elongate catheter shaft 12. This may allow the physician to more easily deliver the catheter system 10 into side branch vessels.

The catheter system 10 may also include a stylet 60 disposed within an internal portion of the elongate catheter shaft 12. The stylet 60 may be arranged and configured to move between a proximal end point 62 and a distal end point 64 within the elongate catheter shaft 12. The proximal end point 62 and the distal end point 64 may be located a predetermined distance from each other, whereby the stylet 60 is free to move within the elongate catheter shaft 12 between the proximal end point 62 and the distal end point 64. Accordingly, when the stylet 60 is moved towards the distal end point 64 the movable tip 54 becomes straightened. When the stylet 60 is moved towards the proximal end point 62 the movable tip 54 may thereby define a curved and/or an angled shape.

The stylet 60 may also be arranged and configured to lock into place with respect to the energy application device 18 and/or the hub 50. In this regard, the movable tip 54 may thereby be locked in its straight, curved, or angled shape when the stylet 60 is also locked in place. Furthermore, in some embodiments, when the stylet 60 is moved to the proximal end point 62 the stylet 60 is completely detached from the internal portion of the elongate catheter shaft 12.

In some embodiments, the energy application device 18 and/or the movable tip 54 may be coated with polymers, or other materials to aid lamination and/or create spacing for wraps. Even still, the energy application device 18 and/or the movable tip 54 may define a longer length (e.g. 15 centimeters, 20 centimeters, and the like) than prior art energy application devices to thereby result in shorter procedure times due to fewer cycles needed to be run. In some embodiments, the stylet 60 may be arranged and configured to allow the elongate catheter shaft 12 to be pushed into and pulled out of the vessel.

### Hub and Proximal Connector Embodiments

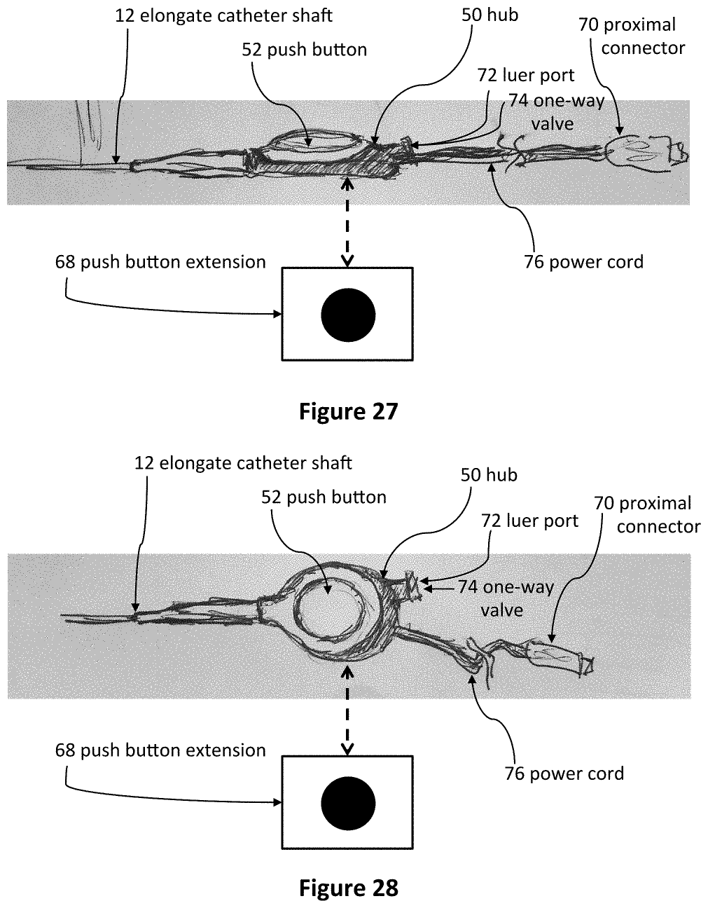

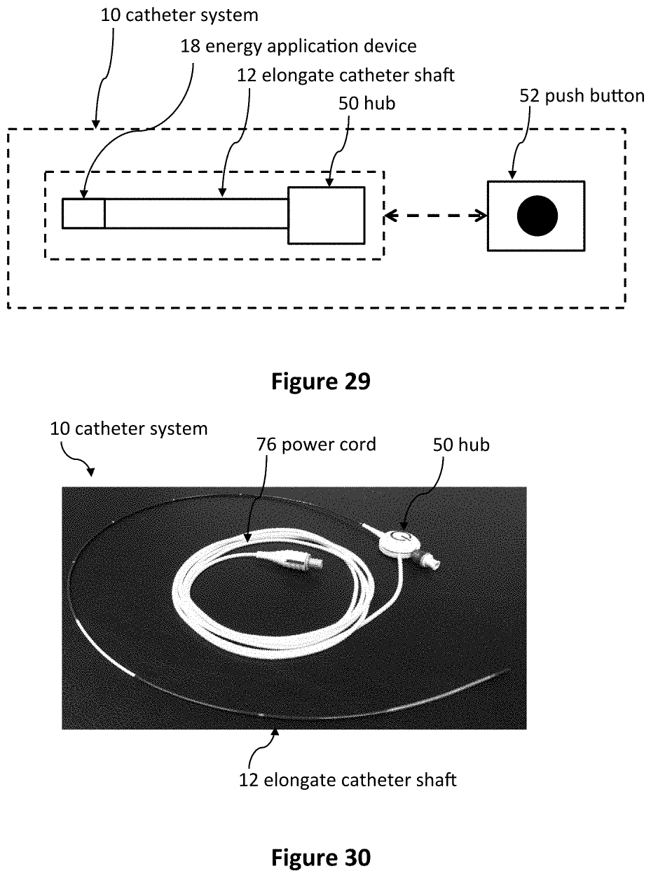

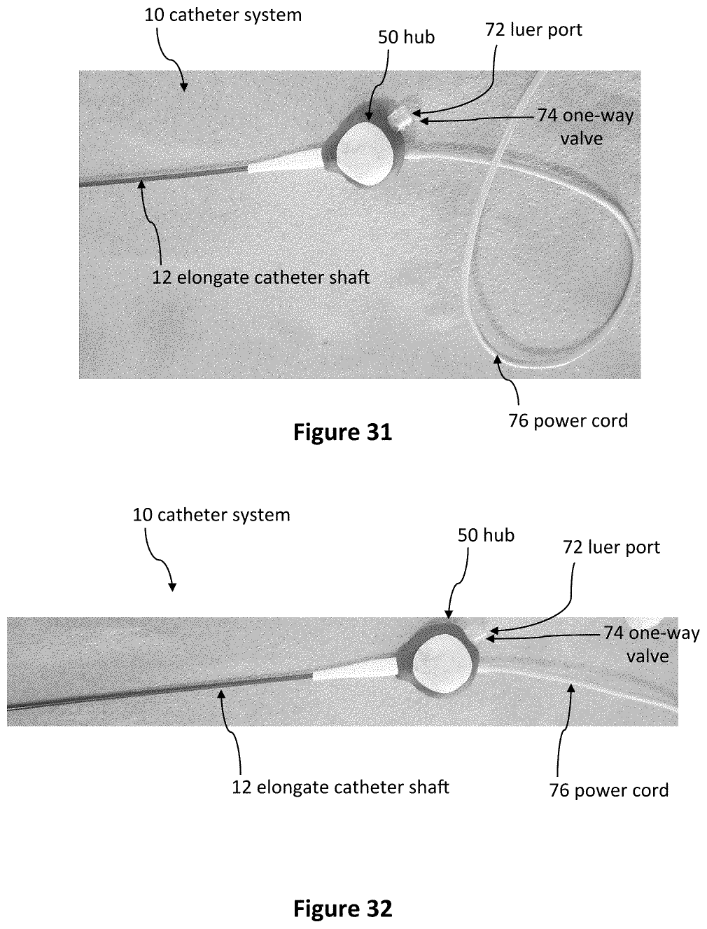

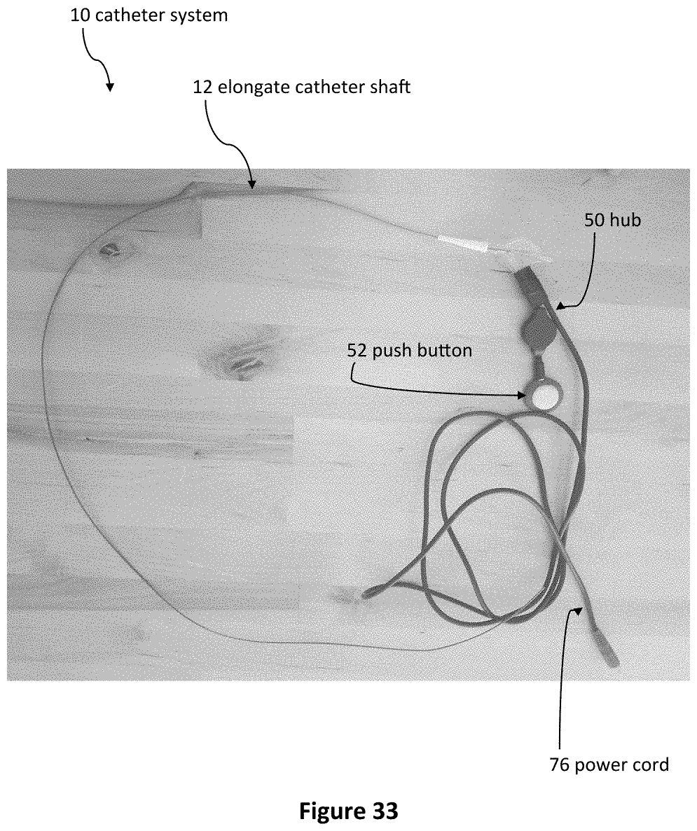

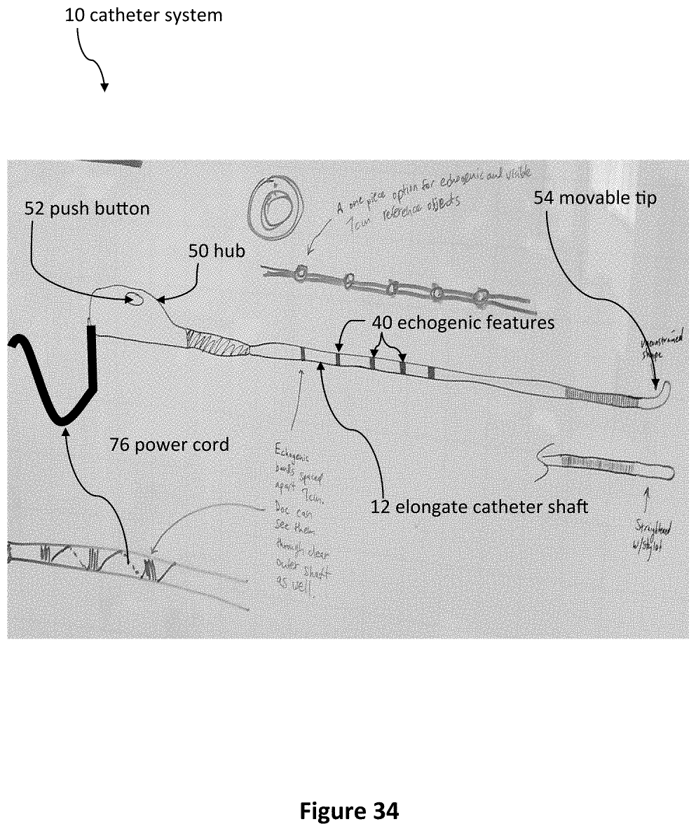

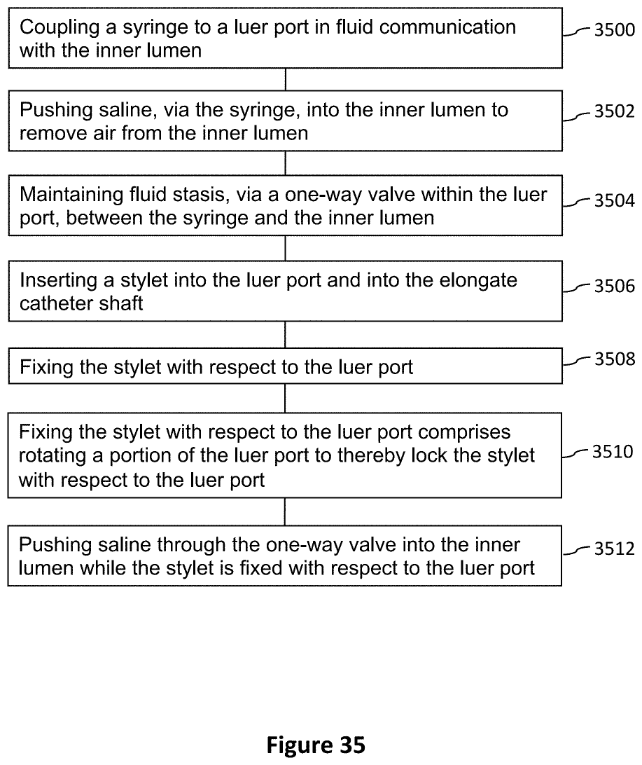

With reference to FIGS. 27-34, the catheter system 10 may include a hub 50 coupled to the proximal end 16 of the elongate catheter shaft 12. The hub 50 may be arranged and configured to provide numerous benefits to improve the treatment process for the physician. As shown in FIG. 27, the hub 50 may be arranged and configured to lay flat on a working surface so that the hub 50 easily couples or smoothly moves along the working surface 22.

The catheter system 10 may also include a push button 52 communicatively coupled to the energy application device 18. The push button 52 may be configured to activate the energy application device 18. As shown in FIGS. 27 and 28, the push button 52 may be coupled to the hub 50. However, in some embodiments, the push button 52 is physically decoupled from the hub 50 and located remotely with respect to the hub 50. Regardless of the push button's location with respect to the hub 50, the push button 52 may still be communicatively and/or electrically coupled to the hub 50. Furthermore, in some embodiments, the hub 50 and push button 52 are sized and shaped to be ergonomically fitted for a human hand to thereby ease the treatment process for the physician.

In some embodiments, the catheter system 10 includes a push button extension 68 communicatively and/or electrically coupled to the energy application device 18, hub 50, and/or push button 52. The push button extension 68 may be located remotely with respect to the hub 50 and push button 52. In some embodiments, the push button extension 68 comprises a wireless remote control. The push button extension 68 may assist the physician because the push button extension 68 may remain in place and not be moved during every movement of the catheter, thus making the treatment faster and easier for the physician. In other words, when the hub 50 and push button 52 move, the push button extension 68 may be arranged and configured to remain in a fixed position with respect to the hub 50 and push button 52. Stated differently, the push button extension 68 may be arranged and configured to move independently with respect to the hub 50 and push button 52.

In some embodiments, the energy application device 18 is electrically coupled to the hub 50. Embodiments may also include a proximal connector 70 that is electrically coupled to the energy application device 18 and the hub 50. The energy application device 18 and the proximal connector 70 may be electrically coupled via a continuous uninterrupted electrical connection, such as a continuous wire.

### Luer Port Embodiments for Prepping the Inner Lumen

The present disclosure also includes embodiments arranged and configured to allow a catheter system 10 to be prepped with saline from a syringe. Accordingly, as shown in FIGS. 27, 28, 31, and 32, the hub 50 may include a luer port 72 arranged and configured to receive the stylet 60. The stylet 60 may thereby be pushed into the luer port 72 whereby the stylet 60 enters and slides along an internal portion of the elongate catheter shaft 12.

Some embodiments may also include a one-way valve 74 in fluid communication with the luer port 72. The one-way valve 74 may be coupled to the stylet 60 whereby the one-way valve 74 is arranged and configured to allow a fluid (e.g. saline) to be discharged into the internal portion of the elongate catheter shaft 12.

Accordingly, the disclosure also includes methods for prepping an inner lumen located within an elongate catheter shaft 12. Methods may include coupling a syringe to a luer port 72 in fluid communication with the inner lumen (at step 3500) and pushing saline, via the syringe, into the inner lumen to remove air from the inner lumen (at step 3502). Methods may also include maintaining fluid stasis, via a one-way valve 74 within the luer port 74, between the syringe and the inner lumen (at step 3504).

Embodiments may also include inserting the stylet 60 into the luer port 72 and into the elongate catheter shaft 12 (at step 3506) and fixing the stylet 60 with respect to the luer port 72 (at step 3508). Fixing the stylet 60 with respect to the luer port 72 may comprise rotating a portion of the luer port 72 to thereby lock the stylet 60 with respect to the luer port 72 (at step 3510). Methods may thereby include pushing saline through the one-way valve 74 into the inner lumen while the stylet 60 is fixed with respect to the luer port 72 (at step 3512).

### Power Cord Embodiments

As shown in FIGS. 27, 28, and 30-34, embodiments disclosed herein may include a power cord 76 coupled to the hub 50 and electrically coupled to the energy application device 18. The power cord 76 may include a braided cover that at least partially surrounds the power cord 76. The power cord 76 may even include a tethered fiber disposed along an inner portion of the power cord 76. The braided cover and/or the tethered fiber may be arranged and configured to provide increased tensile strength so that the power cord 76 will not become damaged when someone pulls on it.

### Interpretation

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section.

Some of the devices, systems, embodiments, and processes use computers. Each of the routines, processes, methods, and algorithms described in the preceding sections may be embodied in, and fully or partially automated by, code modules executed by one or more computers, computer processors, or machines configured to execute computer instructions. The code modules may be stored on any type of non-transitory computer-readable storage medium or tangible computer storage device, such as hard drives, solid state memory, flash memory, optical disc, and/or the like. The processes and algorithms may be implemented partially or wholly in application-specific circuitry. The results of the disclosed processes and process steps may be stored, persistently or otherwise, in any type of non-transitory computer storage such as, e.g., volatile or non-volatile storage.

The various features and processes described above may be used independently of one another, or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments include A, B, and C. The term “and/or” is used to avoid unnecessary redundancy.

Although the embodiments herein are described with various specific embodiments, it will be obvious for a person skilled in the art to practice the invention with modifications. However, all such modifications are deemed to be within the scope of the claims. While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

Furthermore, the foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

### IMPLEMENTATION

The aforementioned embodiments can be implemented, for example, using a machine-readable medium or article which is able to store an instruction or a set of instructions that, if executed by a machine, can cause the machine to perform a method and/or operations described herein. Such machine can include, for example, any suitable processing platform, computing platform, computing device, processing device, electronic device, electronic system, computing system, processing system, computer, processor, or the like, and is able to be implemented using any suitable combination of hardware and/or software.

The machine-readable medium or article can include, for example, any suitable type of memory unit, memory device, memory article, memory medium, storage device, storage article, storage medium and/or storage unit; for example, memory, removable or non-removable media, erasable or non-erasable media, writeable or re-writeable media, digital or analog media, hard disk drive, floppy disk, Compact Disk Read Only Memory (CD-ROM), Compact Disk Recordable (CD-R), Compact Disk Re-Writeable (CD-RW), optical disk, magnetic media, various types of Digital Versatile Disks (DVDs), a tape, a cassette, or the like. The instructions can include any suitable type of code, for example, source code, compiled code, interpreted code, executable code, static code, dynamic code, or the like, and is able to be implemented using any suitable high-level, low-level, object-oriented, visual, compiled and/or interpreted programming language, e.g., C, C #, C++, Java, BASIC, Pascal, Fortran, Cobol, assembly language, machine code, or the like. Functions, operations, components and/or features described herein with reference to one or more embodiments, is able to be combined with, or is able to be utilized in combination with, one or more other functions, operations, components and/or features described herein with reference to one or more other embodiments, or vice versa.

The following is claimed:

1. A catheter system for applying energy to a vessel, comprising:
an elongate catheter shaft having a distal end and a proximal end opposite the distal end;
an energy application device coupled to the distal end of the elongate catheter shaft, wherein the energy application device is configured to apply energy to the vessel; and
a gauge positioned relative to a working surface, wherein the gauge is configured to determine a distance that the elongate catheter shaft travels with respect to a treatment zone.

2. The catheter system of claim 1, wherein the gauge is coupled to the working surface, and the working surface comprises at least one of a patient drape surface, a patient leg surface, a table surface, the elongate catheter shaft, and an introducer sheath that slideably receives the elongate catheter shaft.

3. The catheter system of claim 2, wherein the gauge defines at least one of a tube, rod, and block.

4. The catheter system of claim 2, wherein the gauge defines at least one of a card, sticker, label, and template.

5. The catheter system of claim 4, wherein the gauge is configured to be coupled to the working surface via a hook and loop fastener.

6. The catheter system of claim 4, wherein the gauge is configured to be coupled to the working surface via adhesive.

7. The catheter system of claim 4, wherein the card comprises:
an aperture configured to receive at least one of the introducer sheath and the elongate catheter shaft; and
a slit located adjacent the aperture, wherein the slit is configured such that the aperture removably receives at least one of the elongate catheter shaft and the introducer sheath.

8. The catheter system of claim 4, wherein the gauge comprises at least one marking interval disposed at a predetermined distance.

9. The catheter system of claim 8, wherein the gauge comprises at least one color disposed on the at least one marking interval.

10. The catheter system of claim 8, wherein the energy application device defines a first length and the at least one marking interval is less than or equal to the first length.

11. The catheter system of claim 8, wherein the treatment zone defines a second length and the at least one marking interval is less than or equal to the second length.

12. The catheter system of claim 1, wherein the gauge is slideably coupled to the working surface and the gauge comprises a sliding object that slideably receives at least one of the elongate catheter shaft and the introducer sheath.

13. The catheter system of claim 12, wherein the energy application device defines a first length and the sliding object defines a predetermined length that is less than or equal to the first length.

14. The catheter system of claim 12, wherein the treatment zone defines a second length and the sliding object defines a predetermined length that is less than or equal to the second length.

15. The catheter system of claim 12, wherein the sliding object defines an overall length that is approximately equal to 7 centimeters.

16. The catheter system of claim 12, wherein the sliding object comprises a hollow inner portion that slideably receives the elongate catheter shaft.

17. The catheter system of claim 16, wherein the sliding object slides along a first direction that is parallel to the elongate catheter shaft.

18. The catheter system of claim 16, wherein the sliding object frictionally slides with respect to the elongate catheter shaft along a first direction that is parallel to the elongate catheter shaft, and wherein a friction between the sliding object and the elongate catheter shaft is enough such that the sliding object does not move with respect to the elongate catheter shaft under a force greater than or equal to gravity.

19. The catheter system of claim 16, wherein the sliding object slides along a second direction that is perpendicular to the elongate catheter shaft, and wherein the sliding object clippably couples to the elongate catheter shaft along the second direction.

20. The catheter system of claim 12, wherein the sliding object comprises a variable friction mechanism whereby when the variable friction mechanism is depressed the sliding object slides with respect to the elongate catheter shaft, and when the variable friction mechanism is not depressed the sliding object does not slide with respect to the elongate catheter shaft, and wherein the variable friction mechanism comprises a spring loaded clamping device.
